(19) United States
(12) Patent Application Publication
(10) Pub. No.: US 2016/0346322 A1
(43) Pub. Date: Dec. 1, 2016

(54) SALT WATER GARGLE

(71) Applicant: Kevin Martin, Perrysburg, OH (US)

(72) Inventor: Kevin Martin, Perrysburg, OH (US)

(21) Appl. No.: 15/168,703

(22) Filed: May 31, 2016

Related U.S. Application Data
(60) Provisional application No. 62/169,433, filed on Jun. 1, 2015.

(51) Int. Cl. A61K 33/14; A61K 47/12; A61J 1/14; A61K 47/26

(52) U.S. Cl. CPC A61K 33/14; A61K 47/26; A61K 47/12; A61J 1/1412

(57) ABSTRACT

A kit is provided including a container and a composition disposed in an interior of the container. The composition includes salt, a flavoring agent, and a sweetening agent. The salt is provided wherein when a liquid is added to the composition, the concentration of the salt is from about 0.5% to about 7% by weight. When the liquid is added to the composition, the concentration of the flavoring agent is from about 0.01% to about 1.0% by weight, and the concentration of the sweetening agent is from about 0.001% to about 1.0% by weight. The composition may further include a coloring agent in a concentration from about 0.001% to 1.0% by weight, and a preservative in a concentration from about 0.01% to 2.0% by weight. The composition is provided in a microwave-safe, multi-dose container.

### CROSS-REFERENCE TO RELATED APPLICATIONS

This application claims priority to U.S. Provisional Patent Application Ser. No. 62/169,433, filed Jun. 1, 2015, hereby incorporated by reference in its entirety.

### TECHNICAL FIELD

The disclosure relates to oral rinses, and more particularly, to saltwater solutions for use as a treatment for sore throats.

### BACKGROUND

Sore throats, known medically as pharyngitis, are commonly associated with swelling, inflammation, and pain in the tissues of the throat. This inflammation can be caused by many things. Some common causes include viral or bacterial infections, seasonal allergies, post-nasal drip, dry air, over-use, cough, weather changes, chemicals, et cetera.

Many products and remedies exist for treating sore throats but none are ideal. Multiple mouthwashes are available to gargle with that contain antiseptics and anesthetics, but these are usually very astringent and may contain alcohol which might worsen the throat irritation. Some spray products containing topical numbing agents that numb the throat can be used for pain, but they are difficult to aim, resulting in an unpleasantly numb tongue as well. Cough drops and throat lozenges can be used to provide relief, but they can alter the taste of food and drink horribly afterwards, and represent a potential choking hazard. Tea with honey and lemon is touted as a sore throat remedy, but likely has no more benefit than drinking any other warmed liquid. Some over-the-counter products for gargling might get too hot due to their heating method and represent a potential burn risk. And, if the product is too hot to use right away, this ultimately is inconvenient and time consuming, as you have to wait 5-10 minutes for it to cool before it is safe to use.

People have been gargling with salt water as a home remedy to treat sore throats for years. Gargling with salt water works because water follows salt due to osmosis. For example, gargling a salt water solution and spitting it out causes the back of the throat to be coated with the salt-solution residue. Because the salt water solution has a higher concentration of salt in it than the inflamed tissues, osmosis causes fluid to be pulled from the swollen tissues. This effect causes a decrease in swelling, thereby reducing pain and irritation.

Gargling with salt water works for throat inflammation regardless of the underlying cause. I recommend gargling with saltwater to all my patients with sore throats, but I have encountered a great deal of resistance from people to the remedy for a variety of reasons. I have found the most common reason for peoples' reluctance to gargling with salt water is they do not like the taste.

In addition to the unfavorable taste of a salt water solution, I have discovered many are deterred from the remedy by the inconveniences associated with home mixing and preparation. For example, I have found that people are generally unaware of the optimal concentration of salt in water, wherein too low of a concentration will not be effective, and too high of a concentration will exacerbate the unfavorable taste. I have also discovered that people find the process of mixing a salt water solution can be tedious and messy. I have found that it frustrates people to have to wait for the tap water to warm up, then add salt and mix it with the warmed water solution until it dissolves.

The confusing, inconvenient and labor-intensive way people try gargling with salt water leads to many just giving up on the treatment. Other methods and solutions for treating sore throats fail to cure the aforementioned deficiencies in the art. Generally, other treatments are unpalatable or include active ingredients which are unnecessary and ineffective in the treatment of sore throats.

For example, U.S. Pat. No. 8,709,505 and U.S. Pat. Appl. Ser. No. 2014/0224692 to Boghossian (collectively “Boghossian”) has an inconvenient recommended heating method and because the solution gets too hot, it represents a potential burn risk. Additionally, the composition of Boghossian includes several ingredients which are unnecessary to effectively treat a sore throat, such as vinegar, licorice root, and particularly, antibiotics.

U.S. Pat. Appl. Pub. No. 2014/0294990 to O'Connor similarly fails to cure the deficiencies in the art. For example, O'Conner leaves to chance people using too much or too little water, resulting in unpalatability or a less effective level of salt. Like Boghossian, O'Conner discloses a composition including several ingredients which are unnecessary for the effective treatment of a sore throat, such as aloe vera, Vitamin E, magnesium, zinc, and chamomile, for example.

U.S. Pat. No. 4,457,909 to Tames also fails to provide a palatable and simplified treatment for sore throats. In contrast, Tames is directed towards oral rinsing and bathing, and contemplates a solution including a relatively high concentration of salt and Vitamin C. Additionally, because Tames is directed toward oral health, it does not contemplate sweetening the solution which is important to palatibility.

Another known mixture, sold under the name Simply Gargle, includes a plurality of unnecessary ingredients for treating sore throats, such as grape seed, rosemary, rosehips extract, witch hazel, and sage. Simply Gargle only provides 0.4 ounce individual capsules, which are an ineffective amount for the treatment of a sore throat.

Accordingly, there exists a need in the art for a favorable tasting and convenient salt water solution for use as a treatment for sore throats.

### SUMMARY

In accordance with the instant disclosure, a favorable tasting and convenient salt water solution package and method for use of said solution as a treatment for sore throats, has unexpectedly been discovered.

In a first embodiment, a kit is provided including a container and a composition disposed in an interior of the container. The composition includes salt, a flavoring agent, and a sweetening agent. The salt is provided wherein when a liquid is added to the composition, the concentration of the salt is from about 0.5% to about 7% by weight. When the liquid is added to the composition, the concentration of the flavoring agent is from about 0.01% to about 1.0% by weight, and the concentration of the sweetening agent is from about 0.001% to about 1.0% by weight. The composition may further comprise water, wherein the water is added to a defined point in the container. A preservative and a coloring agent may also be included in the composition.

In another embodiment, an easy to open, prepackaged, microwave-safe sealed plastic cup containing a colored, flavored, and sweetened saltwater solution, at a predetermined concentration of salt and an amount of solution effective for gargling or swishing to soothe minor throat or mouth irritation and relieve pain. One embodiment comprises 4 ounces of a solution with an appropriate concentration of salt and water, in a microwaveable clear plastic cup sealed with an easy to open plastic peel-off lid. The mixture may be colored, flavored and sweetened, and have preservatives added accordingly to enhance visual appeal, taste and freshness prior to sealing the cup during manufacturing. The solution may be heated in a microwave by a user as instructed to an appropriate temperature for user comfort. 4 ounces of solution is enough for four sequences of gargling, swishing and spitting. The combination of the above, especially the convenience and taste, will greatly increase the likelihood of people using the remedy. The solution may be provided in multiple embodiments having different colors, flavors, sweeteners and preservatives for the solution, as well as an embodiment that is uncolored, unflavored and unsweetened. Other embodiments might come in a larger size re-sealable container for multiple uses and users, different sizes and shapes of containers, or a smaller travel-size version to comply with FAA rules for air travel. Other embodiments might have different types of water and different types and concentrations of salt.

In alternate embodiments, a solution may be provided as dissolvable pellets or tablets that can be added to water then placed directly in an affected oral cavity when convenient for the user. In addition, spray and K-cup versions might be envisioned.

Accordingly, several objects and advantages of one or more aspects include: (1) providing a sealed prepackaged plastic container (cup) with a premeasured amount of salt and water solution; (2) providing a solution with an appropriate concentration of salt to be effective without being unpleasant to taste; (3) providing a plastic container (cup) that is microwave safe; (4) providing a plastic container (cup) with an easy-to-open, microwave-safe, plastic peel-off lid; (5) providing a solution that is available in a variety of pleasing flavors such as grape, cherry, lemon, orange, blueberry, raspberry, lime, et cetera or unflavored; (6) providing a solution which can be colored appropriately to match the flavor of the solution for visual appeal for example grape-purple, cherry-red, lemon-yellow, orange-orange, et cetera or uncolored-unflavored; (7) providing a sweetener to enhance the taste of the solution if desired; (8) providing instructions to heat the solution to a desired temperature; (9) providing additives for preserving the solution's color and flavor prior to use; and (10) providing ingredients in a pellet or tablet form to be added to water later. Other objects and advantages will be appreciated by those of ordinary skill in the art.

### DRAWINGS

FIG. 1 is a front perspective view of a kit including a cup and a solution for treatment of a sore throat according to the instant disclosure;

FIG. 2 is a front perspective view of the kit of FIG. 1, wherein a lid of the cup is partially removed;

FIG. 3 is a front perspective view of the kit of FIG. 1, wherein the lid of the cup is fully removed; and

FIG. 4 is a front perspective view of the kit of FIG. 1, wherein a portion of the solution has been discarded from the cup.

### DETAILED DESCRIPTION

The following description of technology is merely exemplary in nature of the subject matter, manufacture, and use of one or more embodiments or aspects, and is not intended to limit the scope, application, or uses of any specific embodiment claimed in this application or in such other applications as may be filed claiming priority to this application, or patents issuing therefrom. Regarding methods disclosed, the order of the steps presented is exemplary in nature, and thus, the order of the steps can be different in various embodiments. Except where otherwise expressly indicated, all numerical quantities in this description are to be understood as modified by the word “about” and all geometric and spatial descriptors are to be understood as modified by the word “substantially” in describing the broadest scope of the technology.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments comprising, consisting of, or consisting essentially of, such materials, components, or process steps including additional materials, components, or processes (for comprising), excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments comprising, consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

In a first embodiment, a composition is provided comprising a solution 10 including at least a salt and water. The water may be any one of a spring water, a distilled water, a filtered water, a bottled water, a tap water, or a combination thereof, for example. Other types of water will be appreciated by those of ordinary skill in the art of food and beverage processing and preparation.

A concentration of the salt in the solution is configured to facilitate the removal of fluid from throat tissues when the solution is gargled for a period of time. The salt concentration may be in the range of 0.5% to 7% by weight of the solution, preferably in the range of 1.0% to 4.0%, and more preferably in the range of 2.0% to 3.5%. However, it has been unexpectedly discovered, after testing, that a salt concentration of about 2.124% is preferred, as salt provided at this concentration is sufficient to actively treat sore throats without subjecting the patient to excessive salinity and unfavorable palatability. Notably, the preferred concentration of salt is comparable to a mixture of about ¾ of a teaspoon of salt in 1 cup of water. In one embodiment, the salt comprises at least one of a sodium chloride or a potassium chloride. It will be appreciated by those of ordinary skill in the art that the salt may be provided as one of several types including, but not limited to an iodized salt, an uniodized salt, a low-sodium salt, a refined table salt, a sea salt, a Himalayan salt, a Kosher salt, and a Celtic salt, or a combination thereof, for example.

To improve palatability, the solution 10 may be appropriately flavored. In one embodiment, the solution 10 may include a flavoring agent comprising a natural flavoring agent, an artificial flavoring agent, or a combination thereof. In the illustrative embodiment, the flavoring agent is provided in a percentage of the total weight of the solution in a range of 0.01% to 1.0%, preferably in the range of 0.03% to 0.3%, and more preferably in a range of 0.05% to 0.1%. It has been unexpectedly discovered, after testing, that this is the most preferable range of sweetener to provide a favorable palatability, wherein the taste of the salt is concealed by the flavoring agent without providing an overwhelming flavor.

The flavoring agent may be any flavor, such as grape, strawberry, orange, cherry, lemon, lime, raspberry, blue-raspberry, strawberry, blueberry, honey, honey-lemon, root beer, jalapeno, peach, banana, apple, apple-cider, mint, green-tea, menthol, vanilla, caramel, fruit punch, ginger, pomegranate, eucalyptus, or a combination thereof, for example. Those of ordinary skill in the art will appreciate that other flavors may be added as desired.

The solution 10 may further include a sweetening agent to improve palatability. The sweetening agent may at least one of a natural sweetening agent and an artificial sweetening agent, such as agave nectar, acesulfame potassium, aspartame, corn syrup, dextrins, dextrose, erythritol, maple syrup, fructose, glucose, galactose, honey, lactose, levulose, maltose, maltodextrins, mannitol, saccharin, sorbitol, stevia, sucralose, sucrose, sugar alcohols, mogrosides, and xylitol, for example. It will also be appreciated that the sweetening agent may comprise a combination of multiple types of sweetening agents. Particularly, it has been unexpectedly discovered, after testing, that sucralose is a preferred sweetening agent due to general consumer acceptance and heat stability. However, other sweetening agents may be utilized in the solution as desired.

A percentage of the sweetening agent by total weight of the solution may be in the range of 0.001% to 1.0%, and preferably in the range of 0.01% to 0.1%, and more preferably in the range of 0.022% to 0.044%. It will be appreciated by those of ordinary skill in the art that different concentrations of sweetening agent may be used depending on the type of sweetening agent used, as each sweetening agent type can have a different level of sweetness. It has been discovered that providing the sweetening agent in the most preferred range is advantageous over other concentrations of sweetening agents, as the concentration of the sweetening agent in the preferred range is sufficient to provide favorable palatability without adversely affecting the treatment of the solution.

The solution 10 may also include a coloring agent configured to add a hue to the solution. The coloring agent could be any food-quality artificial and natural coloring agents, food acceptable quality coloring pigments, dyes, or techniques, known to someone skilled in the art of food and beverage coloring, including but not limited to standard natural and artificial food coloring pigments, liquids, powders, gels, dyes, red 3, red 40, blue 1, yellow 5, and the like, admixed if needed with a vehicle of water and propylene glycol and the like, used alone or in any combination.

The coloring agent may provide a hue in addition to the flavoring agent, including red, purple, yellow, orange, green, lime green, light blue, dark blue, amber, peach, pink, tan, brown, hunter green, light-green, for example. In one embodiment, the hue of the coloring agent is selected to correspond with the selected flavor of the flavoring agent, wherein the coloring agent is representative of a color of a natural form of the flavoring agent. For example, a grape flavoring agent may be paired with a purple coloring agent, and a raspberry flavoring agent may be paired with a red coloring agent. In alternate embodiments, the coloring agent is selected without consideration of the flavoring agent, wherein the hue of the coloring agent may be unrelated to the flavor of the flavoring agent.

The coloring agent is included in the solution in an amount to maximize visual appeal of the solution. For example, including too much of the coloring solution may cause the solution to appear overly concentrated or syrupy, while including too little of the coloring solution may cause the solution to appear watery and ineffective. In one embodiment, the percentage of the coloring agent in the solution by weight may be in a range of 0.001% to 1.0%, and preferably in a range of 0.01% to 0.3%. In some embodiments, the flavoring agent and the coloring agent may be provided as a pre-mixed solution.

The solution may further include a preservative to maintain the color, the flavor and the freshness of the solution 10. In one embodiment, a preservative is a naturally acidic chemical, such as citric acid, malic acid, lactic acid, propionic acid, sodium acid, or other preservatives known to someone skilled in the art of food and beverage preservation, including, but not limited to, ascorbic acid, sulfate, potassium sorbate, potassium citrate, sodium sorbate, sodium citrate, sodium bisulfate, sodium benzoate, benzoic acid, calcium sorbate, parabens, propylparaben, and the like, used alone or in any combination. It has been surprisingly discovered that selection of the preservative may depend on the flavoring agent(s) included in the solution. For example, it has been discovered that malic acid provides favorable flavoring in solution with the grape flavoring agent compared to other types of preservatives. In alternate embodiments, it has been discovered the citric acid is favorable.

The preservative may be provided in the solution in a percentage by weight in a range of 0.01% to 2.0%, and preferably in a range of 0.11% to 0.5%. In the preferred range, the preservative is effective for extending a shelf life of the solution, while minimizing adverse effects to the palatability of the solution.

Although the disclosed solution may comprise the aforementioned ingredients in the disclosed proportions, it has been surprisingly discovered that additional ingredients are unnecessary in the treatment of inflammation of the throat. Accordingly, in one embodiment, the solution will consist essentially of the aforementioned ingredients, wherein additional ingredients which would materially change the composition of the solution and are not disclosed herein are not included in the solution. In yet another embodiment, the solution consists only of the ingredients discussed hereinabove.

Additionally the disclosed proportions of the ingredients were surprisingly found to elicit favorable responses during taste testing. It is commonly believed that increasing the salinity of water will likely result in an unfavorable palatability, as the salt taste of the solution may become overbearing. Accordingly, one would expect an ideal solution to include the lowest concentration of salt possible to effectively treat a sore throat. However, it was discovered that the combination of the salt with the sweetening agent, the flavoring agent, the preservative, and the coloring agent in the disclosed amounts had a counterintuitive result, wherein unexpected amounts of the ingredients worked in synergy to provide a favorable taste, and maximized effectiveness of solution. Particularly, it was discovered that the taste of the solution was more favorable as the salinity of the solution was increased to between about 2.0% and 3.0% by weight. Accordingly, the cooperative nature of the ingredients unexpectedly allowed the salinity of the solution to be maximized without negatively affecting taste.

The following are representative examples of preferred formulations of the solution:

| | | |
|---|---|---|
| | Weight | % Weight (grams per |
| Ingredient | (grams) | 100 milliliter) |

Formulation A

| | | |
|---|---|---|
| Water, distilled | 450.00 | 97.434 |
| Sodium Chloride | 9.80 | 2.122 |
| Citric Acid | 1.50 | 0.325 |
| Cherry Flavor | 0.40 | 0.087 |
| Sucralose, MD | 0.10 | 0.022 |
| Red 3 | 0.05 | 0.011 |

Formulation B

| | | |
|---|---|---|
| Water, distilled | 450.00 | 96.764 |
| Sodium Chloride | 13.00 | 2.795 |
| Citric Acid | 1.50 | 0.323 |
| Cherry Flavor | 0.40 | 0.086 |
| Sucralose, MD | 0.10 | 0.022 |
| Red 3 | 0.05 | 0.011 |

Formulation C

| | | |
|---|---|---|
| Water, distilled | 450.00 | 97.487 |
| Sodium Chloride | 9.80 | 2.123 |
| Citric Acid | 1.20 | 0.260 |
| Natural Cherry Flavor | 0.45 | 0.097 |
| Sucralose, MD | 0.10 | 0.022 |
| Red 40 | 0.05 | 0.011 |

Formulation D

| | | |
|---|---|---|
| Water, distilled | 450.00 | 98.135 |
| Sodium Chloride | 6.50 | 1.418 |
| Citric Acid | 1.50 | 0.327 |
| Natural Cherry Flavor | 0.40 | 0.087 |
| Sucralose, MD | 0.10 | 0.022 |
| Red 40 | 0.05 | 0.011 |

Formulation E

| | | |
|---|---|---|
| Water, distilled | 450.00 | 99.075 |
| Sodium Chloride | 3.30 | 0.727 |
| Citric Acid | 0.50 | 0.110 |
| Natural Cherry Flavor | 0.30 | 0.066 |
| Sucralose, MD | 0.10 | 0.022 |

Formulation G

| | | |
|---|---|---|
| Water, distilled | 450.00 | 98.210 |
| Sodium Chloride | 6.50 | 1.419 |
| Citric Acid | 1.20 | 0.262 |
| Natural Raspberry Flavor | 0.40 | 0.087 |
| Sucralose, MD | 0.10 | 0.022 |

Formulation H

| | | |
|---|---|---|
| Water, distilled | 450.00 | 98.296 |
| Sodium Chloride | 6.50 | 1.420 |
| Citric Acid | 0.80 | 0.175 |
| Natural Raspberry Flavor | 0.40 | 0.087 |
| Sucralose, MD | 0.10 | 0.022 |

Formulation I

| | | |
|---|---|---|
| Water, distilled | 450.00 | 98.146 |
| Sodium Chloride | 6.50 | 1.418 |
| Citric Acid | 1.50 | 0.327 |
| Natural Raspberry Flavor | 0.40 | 0.087 |
| Sucralose, MD | 0.10 | 0.022 |

Formulation J

| | | |
|---|---|---|
| Water, distilled | 450.00 | 98.146 |
| Sodium Chloride | 6.50 | 1.418 |
| Citric Acid | 1.50 | 0.327 |
| Natural Raspberry Flavor | 0.40 | 0.087 |
| Sucralose, MD | 0.10 | 0.022 |

Formulation K

| | | |
|---|---|---|
| Water, distilled | 450.00 | 99.075 |
| Sodium Chloride | 3.30 | 0.727 |
| Citric Acid | 0.50 | 0.110 |
| Natural Lemon Lime Flavor | 0.20 | 0.044 |
| Sucralose, MD | 0.20 | 0.044 |

Formulation L

| | | |
|---|---|---|
| Water, distilled | 450.00 | 99.075 |
| Sodium Chloride | 3.30 | 0.727 |
| Citric Acid | 0.50 | 0.110 |
| Natural Lemon Lime Flavor | 0.30 | 0.066 |
| Sucralose, MD | 0.10 | 0.022 |

Formulation M

| | | |
|---|---|---|
| Water, distilled | 450.00 | 99.075 |
| Sodium Chloride | 3.30 | 0.727 |
| Citric Acid | 0.50 | 0.110 |
| Orange Zest Flavor | 0.30 | 0.066 |
| Sucralose, MD | 0.10 | 0.022 |

Formulation N

| | | |
|---|---|---|
| Water, distilled | 450.00 | 99.053 |
| Sodium Chloride | 3.30 | 0.726 |
| Citric Acid | 0.50 | 0.110 |
| Natural and Artificial Orange | 0.30 | 0.066 |
| Flavor | | |
| Sucralose, MD | 0.20 | 0.044 |

Formulation O

| | | |
|---|---|---|
| Water, distilled | 450.00 | 99.043 |
| Sodium Chloride | 3.30 | 0.726 |
| Citric Acid | 0.50 | 0.110 |
| Natural and Artificial | 0.45 | 0.099 |
| Orange Flavor | | |
| Sucralose, MD | 0.10 | 0.022 |

Formulation P

| | | |
|---|---|---|
| Water, distilled | 450.00 | 98.912 |
| Sodium Chloride | 3.30 | 0.725 |
| Citric Acid | 1.00 | 0.220 |
| Natural and Artificial | 0.40 | 0.088 |
| Orange Flavor | | |
| Sucralose, MD | 0.10 | 0.022 |
| Yellow 5 | 0.10 | 0.022 |
| Red 3 | 0.05 | 0.011 |

Formulation Q

| | | |
|---|---|---|
| Water, distilled | 450.00 | 98.178 |
| Sodium Chloride | 6.50 | 1.418 |
| Citric Acid | 1.20 | 0.262 |
| Natural and Artificial | 0.40 | 0.087 |
| Orange Flavor | | |
| Sucralose, MD | 0.10 | 0.022 |
| Yellow 5 | 0.10 | 0.022 |
| Red 3 | 0.05 | 0.011 |

Formulation R

| | | |
|---|---|---|
| Water, distilled | 450.00 | 97.476 |
| Sodium Chloride | 9.80 | 2.123 |
| Citric Acid | 1.20 | 0.260 |
| Natural and Artificial Orange | 0.40 | 0.087 |
| Flavor | | |
| Sucralose, MD | 0.10 | 0.022 |
| Yellow 5 | 0.10 | 0.022 |
| Red 3 | 0.05 | 0.011 |

Formulation S

| | | |
|---|---|---|
| Water, distilled | 450.00 | 99.075 |
| Sodium Chloride | 3.30 | 0.727 |
| Citric Acid | 0.50 | 0.110 |
| Natural Grape Flavor | 0.30 | 0.066 |
| Sucralose, MD | 0.10 | 0.022 |

Formulation T

| | | |
|---|---|---|
| Water, distilled | 450.00 | 99.075 |
| Sodium Chloride | 3.30 | 0.727 |
| Citric Acid | 0.50 | 0.110 |
| Natural Strawberry Flavor | 0.30 | 0.066 |
| Sucralose, MD | 0.10 | 0.022 |

Formulation U

| | | |
|---|---|---|
| Water, distilled | 450.00 | 99.075 |
| Sodium Chloride | 3.30 | 0.727 |
| Citric Acid | 0.50 | 0.110 |
| Natural and Artificial Peach | 0.30 | 0.066 |
| Flavoring | | |
| Sucralose, MD | 0.10 | 0.022 |

The solution is prepared by mixing each of the ingredients together at a temperature greater than room temperature. It has been surprisingly discovered that mixing the ingredients and heating to a temperature between 150 degrees and 220 degrees Fahrenheit, and more preferably, mixing and then heating the ingredients to a temperature of 180-185 degrees Fahrenheit provides favorable results. Alternatively, depending on food safety and processing requirements, known to those skilled in the art of food and beverage preparation, the ingredients may not need to be heated during mixing.

Although the illustrated embodiment includes a solution pre-mixed with water, it will be appreciated that the composition may be provided in a dry form, such as a powder, pellet, or tablet comprising the salt, the coloring agent, the flavoring agent, the preservative and the sweetening agent, without the water. In the dry form, the ingredients may be formed with known binding agents, diluents, pigments, flow aids, glidants, and lubricants, used in the proper proportions known to one skilled in the art of the food and tablet manufacturing industries to create a tablet or pellet using techniques known in the medicinal packaging industry and food packaging, processing and preparation industry. Particularly, the ingredients of the dry form of the composition may be provided in the proportions provided in formulations A-U above, wherein the water is not included in the solution.

The currently contemplated binding agents used to aid in tablet formation are microcrystalline cellulose and magnesium stearate, but could be any known artificial and natural binding or thickening agents of food acceptable quality including but not limited to poly dextrose, dextrins, maltodextrins, natural gums, xanthum gum, gelatins, corn starch, modified starches, cellulose derivatives and the like, used alone or in any combination.

In forming the tablet, the ingredients could also be combined with dry sodium bicarbonate and dry citric acid powder in the proper proportions known to one skilled in the art of tablet manufacturing to create an effervescent tablet which would be reconstituted in water prior to use.

The above contemplated tablet embodiment, as well as others, could be available with a coating agent applied to protect the tablet during packaging and shipping but still allow dissolubility in water when ready for use. Any tablet coating agents of food acceptable quality known in the food, medicinal, pharmaceutical and manufacturing industries including but not limited to polymers, polysaccharides, plasticizers and pigments and the like, used alone or in any combination could be added to the tablets.

In yet another embodiment, the composition may be provided in connection with a capsule for use in a conventional coffee maker, such as a K-cup, for example. In this embodiment, the desired ingredients would be provided within the cup, wherein when the capsule is placed in a coffee maker, heated water is passed through the capsule to create the solution 10 as described hereinabove.

In yet another embodiment, the composition may be provided in the form of a dissolvable strip, wherein the strip comprises at least the salt, the flavoring agent, and the coloring agent. The strip is configured wherein when the strip is placed in the oral cavity of a user, and particularly, on a tongue of the user, the strip dissolves to form the solution 10. The solution 10 may then be gargled and spit out by the user. The user may add a dose of water as desired to dilute the concentration of the solution 10. Similarly, the composition may be provided as part of a dissolvable chew, or in a chewing gum.

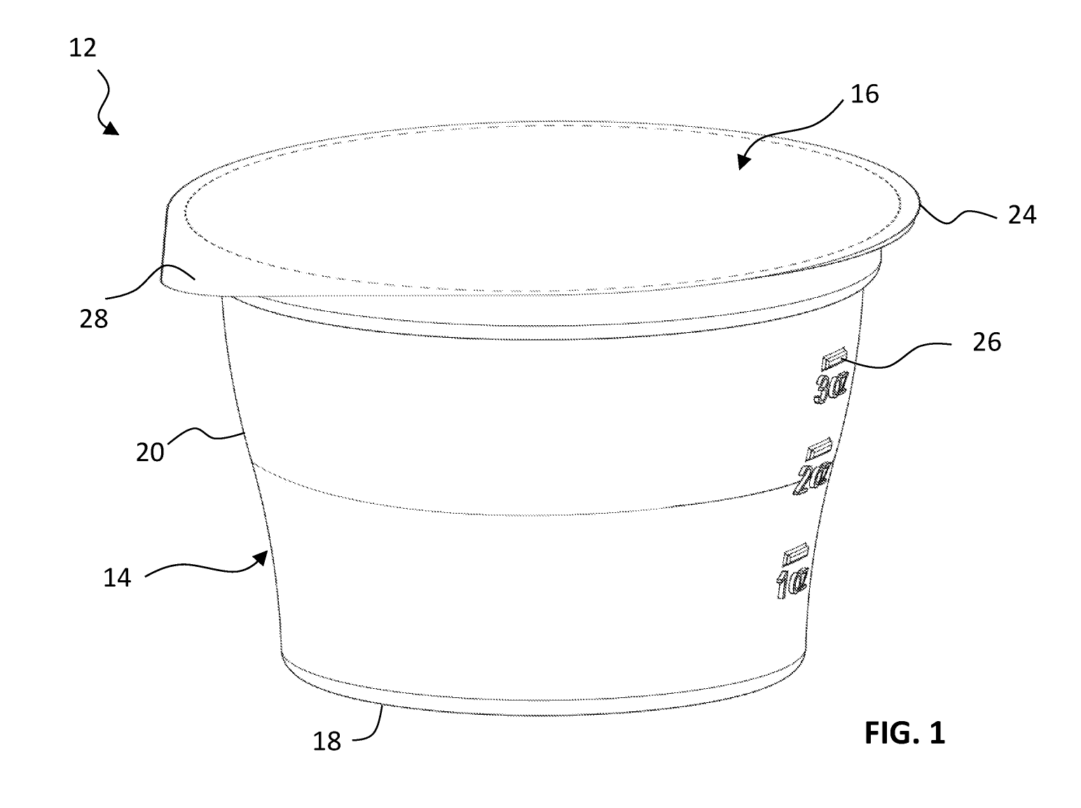

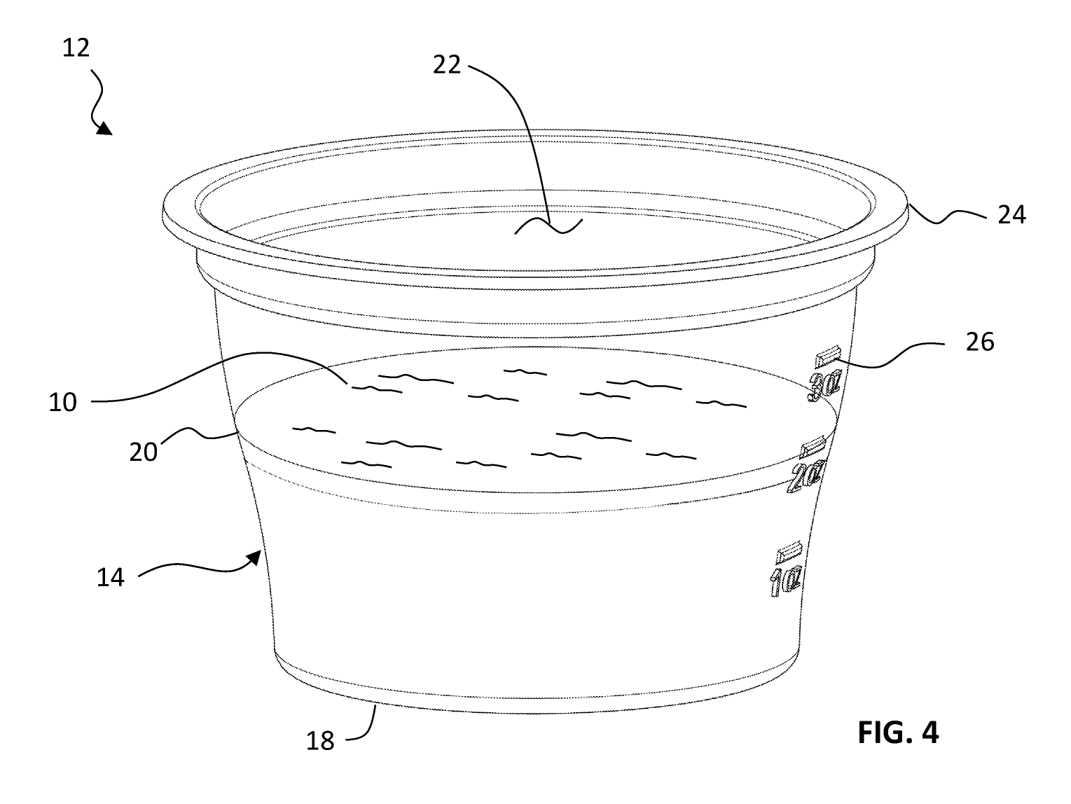

FIGS. 1-4 show a kit 12 according to a first embodiment of the instant disclosure. As shown in FIG. 1, the kit 12 includes a cup 14 having a removable lid 16, wherein the solution 10 is contained within the cup 14. In the illustrated embodiment, the cup 14 is monolithically formed of a polymeric material. The cup 14 may be formed using known processes for molding polymeric materials, such as rotational molding, blow molding, injection molding, and compression molding, for example. The cup 14 is constructed in the usual manner by one skilled in the art of manufacturing plastic containers in the food and beverage processing and packaging industry, in accordance with the embodiment.

In the illustrated embodiment, the cup 14 includes a bottom 18 and a continuously formed sidewall 20 extending from the bottom 18 to define an interior cavity 22 of the cup 14. As shown, the sidewall 20 tapers outwardly from the bottom of the cup 14. In alternate embodiments, the sidewall 20 may be formed orthogonally to the bottom 18, or taper inwardly from the bottom 18. The sidewall 20 further includes a rim 24 extending radially outwardly therefrom, wherein the rim 24 is formed adjacent an opening into the cavity 22 of the cup 14, and forms a planar sealing surface.

The cup 14 further includes a series of graduations 26 formed in the sidewall 20, wherein each of the graduations 26 indicates a predetermined volume of the cavity 22 of the cup 14. For example, the cup 14 may include graduations 26 indicating one-ounce increments in the volume of the cup 14. It will be appreciated that other increments and units of measurement may be used.

In alternate embodiments, the cup 14 may be formed with features to maximize ergonomics, such as indentations or protuberances configured to accommodate fingers. It may also have temperature sensitive colormetric tape (not shown) on the side of the cup 14 so a user can determine the temperature of the solution 10 within the cup 14.

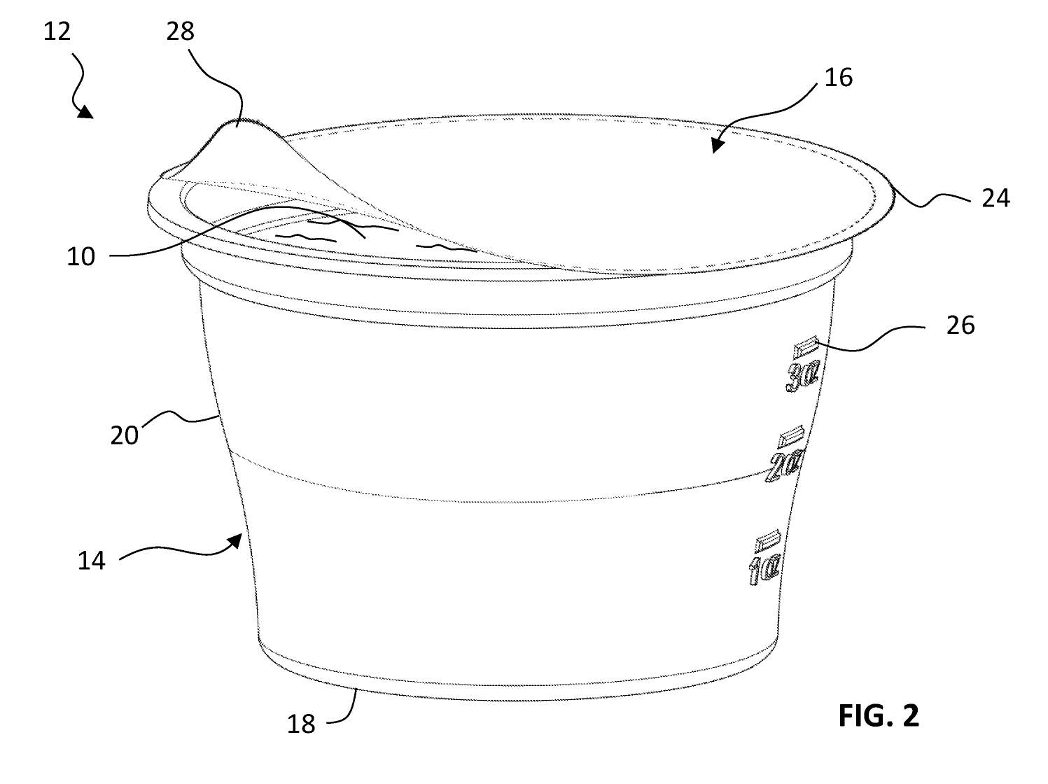

Referring to FIGS. 1 and 2, the lid 16 is hermetically sealed to the rim 24 of the cup 14, and is configured to be peeled from the cup 14. The lid 16 may constructed in the usual manner by one skilled in the art of manufacturing lids to seal plastic containers for holding food and liquid in the food processing and packaging industry, in accordance with the embodiment to be placed and sealed on the top of the cup 14 using sealing techniques, known by those skilled in the art of sealing containers for food and beverage, including, but not limited to, heat sealing, vacuum sealing, lamination, and food and microwave-safe adhesives. The lid 16 may include a tab 28 formed thereon, wherein the tab 28 extends radially outwardly from the rim 24 of the cup 24 when the lid 16 is assembled thereto, thereby providing a portion of the lid 16 to be grasped by a user to facilitate peeling of the lid 16 from the cup 14, as shown in FIG. 2.

Referring again to FIG. 1, the solution 10 is hermetically sealed within the interior cavity 22 of cup 14 by the lid 16, wherein the interior cavity 22 is impervious to water, water vapor, air, and other gases. The solution 10 may be provided to the interior cavity 22 using a process for achieving commercial sterility in the kit 12, including a hot fill process, pasteurization, high-pressure processing, ultraviolet radiation, or a combination thereof, for example. Other methods or combinations of methods for achieving commercial sterility will be appreciated by those skilled in the art.

Although a 4 ounce cup 14 is illustrated, it will be appreciated that the cup 14 may be provided in any size or shape suitable to hold or dispense the solution 10. For example, a size of the cup 14 may be increased to minimize the chance of spilling the solution 10. The cup 14 may be a different shape, such as a square-cube, cylindrical, rectangular, or a pouch. The cup 14 could be taller or shorter, wider or narrower.

In one embodiment, the cup 14 and lid 16 are made of clear plastic. However, it will be appreciated that the cup 14 and lid 16 could be made of other materials, such as paper, foam, or a metallic foil, for example.

In other embodiments of the kit 12, the solution 10 may be provided in bulk-sized, resealable containers, wherein after use of a portion of the solution, a remainder of the solution can be placed in storage for later use. For example, a 1 quart size bottle of the solution for multiple uses and users could be available and people could use their own cups to pour in the solution 10, pellets, or tablets. The remaining portion of the solution 10 could be refrigerated or stored after opening. Conversely, the solution 10, pellet, and tablet embodiments could come in smaller travel sizes. For example 1-2 ounce packages, wherein the solution 10 could would be compliant with FAA travel restrictions on fluids.

Referring again to FIGS. 1-4, a method for treating a sore throat using the solution 10 is disclosed. The method includes the steps of: retrieving the kit 12 including the solution 10 and the cup 14; shaking the cup 14 to mix the solution 10; venting the cup 14 to prevent the lid 16 from bursting open during heating; heating the solution 10; removing the lid 16 from the cup 14; testing the temperature of the solution 10; and gargling a portion of the solution 10 to treat the affected sore throat. The steps of the method may be performed in the sequence provided, or in alternate sequences. Additionally, some of the steps of the method may be repeated multiple times, or skipped entirely.

In a first step, the kit 12 is retrieved by an affected user or someone caring for the affected user; for safety purposes, parents should prepare and test the solution if it is to be used by a child. The solution 10 may then be mixed in the cup 14 to ensure homogenous distribution of the ingredients therein. In one embodiment, the solution 10 is mixed by shaking the sealed cup 14 for approximately 5 seconds. However, it will be appreciated that the lid 16 of the cup 14 may be removed, wherein the solution 10 can be stirred to facilitate mixing.

The solution 10 may then be heated to improve solubility of the ingredients in the water, and to improve palatability of the solution. In one embodiment, lid 16 of the cup 14 may be partially peeled from the rim 24 of the cup 14, wherein a vent is formed into the interior cavity 22 of the cup 14, as shown in FIG. 2. The cup 14 containing the solution 10 is then placed upright in a microwave for a period of time. In one embodiment, the solution 10 is heated to a temperature of between 106 and 110 degrees Fahrenheit. It has been discovered that 4 ounces of the solution 10 stored at room temperature, will get heated to between 106 and 110 degrees Fahrenheit in about 10-20 seconds, depending on the output power of the microwave. It will be appreciated that in alternate embodiments of the method, the solution 10 may not be heated, or may be chilled, depending on a preference of the user.

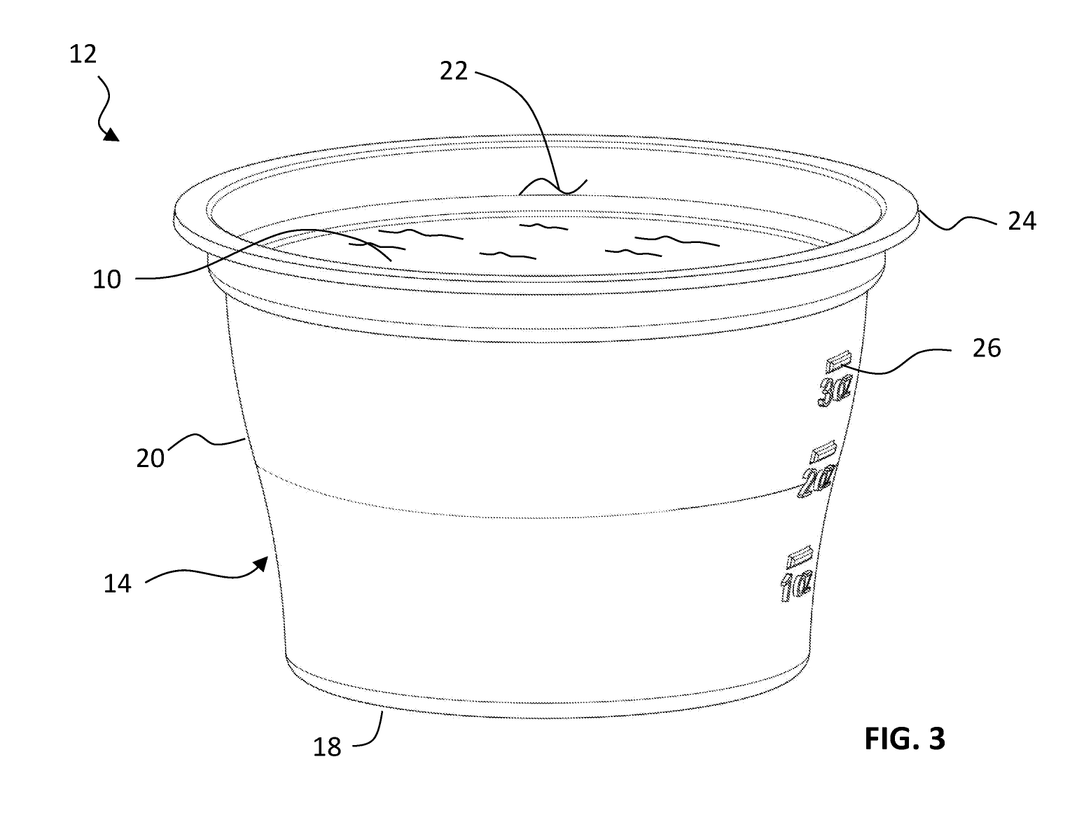

After heating, the sealed cup 14 containing the solution 10 should first be felt to ensure it is not too hot to the touch. If the cup 14 is too hot to handle, it can be left in the microwave to cool. Once safe, the cup 14 can be removed from the microwave and placed upright on a counter or other flat, solid surface. With the bottom 18 of the cup 14 sitting on the flat surface, the user can hold the sidewall 20 of the cup 14 in one hand and can grasp the tab 28 of the lid 16 with the thumb and index finger of the opposite hand to completely remove the lid 16 from the cup 14, as shown in FIG. 3.

After removal of the lid 16, the temperature of the solution 10 should again be tested by the user prior to use by gently placing a clean fingertip into the solution 10 to ensure it is not too hot. If the user feels it is too warm to use at this point, the solution can be allowed to cool for a period of time and then retested.

With the solution 10 properly heated and mixed, approximately 1 ounce of the solution 10 is poured into an affected oral cavity of the user. The solution 10 is then gargled in a usual manner for 5-10 seconds, wherein the solution 10 will rinse the throat of the affected used. The gargled solution 10 is then discarded without swallowing. It is contemplated in the first embodiment that 4 ounces of solution is enough for 4 sequences of gargling and spitting. Thus the aforementioned gargling step is recommended to be repeated three additional times over the course of about 1 minute for maximized effect.

After four sequences of gargling and discarding the solution 10, the cup 14 and lid 16 should be discarded or recycled after use. Treatment may be repeated as needed.

In an alternate embodiment of the method, the dry combination of the ingredients can be combined with water by the user prior to mixing. In this embodiment, the water may be heated prior to adding the dry form of the solution to improve solubility of the ingredients of the solution.

In yet another embodiment of the method, the dry combination of the ingredients could also be placed directly into the mouth of the user, allowed to dissolve and be either gargled or swished, and discarded after use.

In yet another embodiment, the solution may be provided as a concentrated liquid form, available as a spray version, wherein the amount of the water in the solution is minimized. In the concentrated form, the solution could be sprayed directly into the user's mouth. The user may then sip some water, gargle the solution, and spit it out, as described hereinabove.

Thus the reader will see that some embodiments may come in a variety of different salts, different waters, different colors, different flavors, different concentrations, different sizes, different ingredients, and different delivery methods and be used at different temperatures to suit the user's needs and tastes. Accordingly, the scope should be determined not by the embodiments mentioned and illustrated, but by the appended claims and their legal equivalents. From the foregoing description, one ordinarily skilled in the art can easily ascertain the essential characteristics of this invention and, without departing from the spirit and scope thereof, can make various changes and modifications to the invention to adapt it to various usages and conditions.

1. A kit for treatment of a sore throat comprising:
a container; and
a composition disposed in an interior of the container, the composition including a salt, a flavoring agent, and a sweetening agent, where adding a liquid to a defined point of the container results in a concentration of the salt from about 0.5% to about 7% by weight, a concentration of the flavoring agent from about 0.01% to about 1.0% by weight, and a concentration of the sweetening agent from about 0.001% to about 1.0% by weight.

2. The kit of claim 1, wherein the composition further comprises water.

3. The kit of claim 1, wherein the composition further comprises water and the water is added to the defined point of the container.

4. The kit of claim 1, where adding the liquid to the defined point of the container results in a concentration of the salt from about 2.0% to about 3.5% by weight.

5. The kit of claim 1, wherein the salt is a member of the group consisting of sodium chloride, potassium chloride, sodium salts, potassium salts, and combinations thereof.

6. The kit of claim 1, wherein the composition further comprises a preservative, where filling the container with the liquid to the defined point of the container results in a concentration of the preservative from about 0.01% to about 2.0% by weight.

7. The kit of claim 1, wherein the composition further comprises a coloring agent, where filling the container with the liquid to the defined point of the container results in a concentration of the coloring agent from about 0.001% to about 1.0% by weight.

8. The kit of claim 1, where filling the container with the liquid to the defined point of the container results in a concentration of the flavoring agent from about 0.03% to about 0.3% by weight.

9. The kit of claim 1, where filling the container with the liquid to the defined point of the container results in a concentration of the sweetening agent from about 0.02% to about 0.044% by weight.

10. The kit of claim 1, wherein the composition consists essentially of the salt, the flavoring agent, and the sweetening agent.

11. The kit of claim 1, wherein the container comprises a cup with a removable lid.

12. The kit of claim 1, wherein the container comprises a plurality of graduations formed on a sidewall thereof, the plurality of graduations including the defined point.

13. A method of preparing a treatment for a sore throat comprising:
providing a kit for treatment of a sore throat, the kit including a container and a composition disposed in an interior of the container, the composition including a salt, a flavoring agent, and a sweetening agent, where adding a liquid to a defined point of the container results in a concentration of the salt from about 0.5% to about 7% by weight, a concentration of the flavoring agent from about 0.01% to about 1.0% by weight, and a concentration of the sweetening agent from about 0.001% to about 1.0% by weight; and
adding a liquid to the defined point of the container.

14. The method of claim 13, wherein the liquid comprises water.

15. The method of claim 13, further comprising mixing the liquid and the composition.

16. The method of claim 13, further comprising heating the liquid and the composition.

17. A method of treating a subject having a sore throat comprising:
preparing a treatment for a sore throat according to the method of claim 13; and
administering the treatment for the sore throat by having the subject gargle at least a portion of the liquid added to the defined point of the container.

18. A method of treating a subject having a sore throat comprising:
providing a kit for treatment of a sore throat, the kit including a container and a composition disposed in an interior of the container, the composition including a salt, a flavoring agent, a sweetening agent, and water providing a concentration of the salt from about 0.5% to about 7% by weight, a concentration of the flavoring agent from about 0.01% to about 1.0% by weight, and a concentration of the sweetening agent from about 0.001% to about 1.0% by weight; and
administering the treatment for the sore throat by having the subject gargle at least a portion of the composition.

19. The method of claim 18, further comprising heating the composition prior to the administering step.

20. The method of claim 18, further comprising repeating the administering step.
